(19) United States
(12) Patent Application Publication
(10) Pub. No.: US 2005/0043757 A1
(43) Pub. Date: Feb. 24, 2005

(54) Medical devices formed from shape memory alloys displaying a stress-retained martensitic state and method for use thereof

(76) Inventors: Michael Arad, Tel Aviv (IL); Leonid Monassevitch, Hadera (IL); Amir Perle, Haifa (IL); Noa BenDov-Laks, Tel Aviv (IL)

Correspondence Address: PEARNE & GORDON LLP, 1801 EAST 9TH STREET, SUITE 1200, CLEVELAND, OH, 44114-3108, US

(21) Appl. No.: 10/903,823

(22) Filed: Jul. 30, 2004

Related U.S. Application Data
(63) Continuation-in-part of application No. 10/158,673, filed on May 30, 2002.
Application No. 10/158,673 is a continuation-in-part of application No. 09/592,518, filed on Jun. 12, 2000, now Pat. No. 6,402,765.

(51) Int. Cl. A61M 29/00

(52) U.S. Cl. 606/200

(57) ABSTRACT

A method is disclosed for utilizing a deformable article of manufacture formed at least partly of a shape memory alloy. The method includes the steps of deforming the article from a first predetermined configuration to a second predetermined configuration while the shape memory alloy is, at least partially, in its stable martensitic state and at a first temperature. A resisting force is applied to the deformed article of manufacture using a restraining means and the article is heated from the first temperature to a second temperature in the presence of the resisting force. The stable martensitic state is transformed to a metastable stress-retained martensitic state. The resisting force is then removed allowing the alloy to transform to its austenitic state and the shape of the article to be restored substantially to its first configuration. Devices primarily medical devices operative by employing this method are also disclosed.

### REFERENCE TO CO-PENDING APPLICATIONS

The present application is a continuation-in-part of U.S. application Ser. No. 10/158,673, entitled “Surgical Clip Applicator Device”, filed May 30, 2002, which is itself a continuation-in-part of U.S. application Ser. No. 09/592,518, entitled “Surgical Clips”, filed Jun. 12, 2000. The contents of both of these applications are incorporated by reference herein.

### FIELD OF THE INVENTION

The present invention relates to devices and, more specifically, to medical devices formed from shape memory alloys and a method for use thereof.

### Glossary and Symbols

Austenite—high temperature, high symmetry phase. In what is discussed herein the austenitic phase includes structures such as the B2 and R structures.

Martensite—low temperature, low symmetry phase. This phase has a different microstructure from that of the austenite phase, but a specimen, i.e device, in this state has substantially the same external shape as it does in the austenite state. This state may also be referred to herein as undeformed or cooling-induced martensite, the terms being used interchangeably without any attempt at distinguishing between them.

Deformed martensite—A martensitic state having a microstructure different from that of undeformed martensite. Devices formed from alloys in this state have an external shape different from their external shape when the alloy is in its undeformed martensitic state.

Martensitic transformation—diffusionless phase transformation of austenite to martensite. The reverse martensitic transformation as used herein is the phase transformation wherein martensite is transformed into austenite.

Mₛ—temperature at which the martensitic transformation begins.

M_{f}—temperature at which the martensitic transformation is completed.

Aₛ—temperature at which the reverse martensitic transformation phase begins.

A_{f}—temperature at which the reverse martensitic transformation is completed with the alloy being completely austenitic.

M_{d}—maximum temperature at which it is possible to obtain stress-induced martensite (SIM) or to maintain stress-retained martensite. (SRM)

SMA—shape memory alloy—An alloy that inter alia has SME, SE, and SEP properties allowing it to recover its original shape after large deformations. A typical, but non-limiting, example of SMAs are nickel-titanium alloys.

SME—shape memory effect—A property of SMA where the alloy recovers its original shape upon heating. This effect can occur only if the alloy is deformed at temperatures below A_{f}.

SE—superelasticity effect—A property of SMA where the alloy recovers its original shape upon unloading, typically, but not necessarily, at isothermal conditions. This effect can occur only if the alloy is deformed and unloaded at temperatures above A_{f}. This effect is frequently also called pseudoelasticity.

SEP—superelastic plasticity effect—A property of SMA where the alloy recovers its original shape upon unloading, typically, but not necessarily, at isothermal conditions. This effect can occur only if the alloy is deformed at temperatures below A_{f} and unloaded at temperatures above A_{f}.

SRM—stress-retained martensite—a deformed metastable martensitic state obtained by deformation of martensite at temperatures below A_{f} and by retaining the deformed state by applying a restraining means at temperatures above A_{f}.

SIM—stress-induced martensite—a deformed martensitic state obtained by deformation of austenite at temperatures above Mₛ.

In the discussion below, the terms “phase” and “state” will be used interchangeably with no intention at distinguishing between them.

### BACKGROUND OF THE INVENTION

Metals and metal alloys having shape memory characteristics are known in the art. Shape memory alloys (SMA) may exhibit both a shape memory effect (SME) and a superelasticity effect (SE). Phenomenologically, SME occurs when a device formed from an SMA is deformed at a reduced temperature with the device returning to its original shape upon heating. SE occurs when a device, formed from an SMA, is deformed under a load; the device recovers its original shape upon removal of the load without a change in temperature. The recovery mechanisms of SME and SE are both associated with a reversible martensitic transformation. In the case of SME, recovery occurs after heating, while in the case of SE, recovery occurs after removing a load.

A device made from a shape memory alloy (SMA) is relatively easily deformed from its original shape to a new shape when cooled below the temperature at which the alloy is transformed from its austenitic to its martensitic state. Referring now to FIG. 1, the fully austenitic phase is present at or above temperature A_{f}. While cooling from or above A_{f}, the temperature at which the alloy begins its transformation from the austenitic state to the martensitic state is referred to as Mₛ. The temperature at which this transformation is complete is denoted as M_{f}, at, and below, M_{f} only the martensitic phase is present. Between Mₛ and M_{f}, both martensitic and austenitic phases exist.

As seen in FIG. 1, when a device made from a SMA is warmed from or below temperature M_{f}, the alloy starts to revert to its austenitic state at a temperature Aₛ. At a temperature A_{f}, the reversion is complete, and the alloy is 100% austenitic.

The curves in FIG. 1 represent the reversible martensitic transformation which determines the shape memory effect (SME) discussed above. In FIG. 1, M_{d} represents the temperature at or above which no martensite can exist, regardless of the application of a distorting force.

Between the temperatures M_{f} and A_{f} shown in FIG. 1 the alloy may contain either 100% austenite or 100% martensite or a mixture of austenite and martensite. As discussed above, the state (states) that exists (exist) in this temperature range will depend on whether the temperature change is effected from above A_{f} or below M_{f} respectively, as well as the magnitude of the temperature change. This is a result of hysteresis in the martensitic transformation.

Referring now to FIG. 2 there is illustrated a schematic representation of the phase transformations occurring in a shape memory alloy subjected to controlled stress and temperature changes. Region 10 represents the stable martensitic phase and region 12 represents the metastable martensitic phase. The Clausius-Clapeyron (CC) relationship 14 separates the stable austenitic phase region 16 from the metastable martensitic phase region 12. The CC relationship 14 represents the critical stress required to induce martensite as a function of temperature.

Reference is now made to FIGS. 3A and 3B. FIG. 3A schematically illustrates the shape memory effect (SME) in a stress versus temperature diagram. In FIG. 3A, a device formed from a SMA is initially cooled 20 from above temperature Af, where the alloy is fully austenitic, to below M. where the alloy starts its transition to the martensitic state. The cooled device is then plastically deformed 22 by a stress. When the deforming force is removed 24, the device retains its deformed shape as indicated by the parallelogram-like shape in the Figure. Heating 26 the device to above temperature A_{f} results in a phase transition to 100% austenite and the device reverts substantially to its original shape.

FIG. 3B is an alternative method of using the shape memory effect (SME). The device shown is formed from an SMA at a temperature above Mₛ and below A_{f}, where the alloy is in its fully austenitic state. The austenite is stressed 27 to form deformed martensite (stress-induced martensite). The device remains in its deformed state after removing 28 the load. When heated 29 above A_{f}, a phase transition occurs and the alloy transforms to 100% austenite with the device reverting substantially to its original shape.

In FIGS. 3A and 3B, as well as FIGS. 5, 6 and 7 to be discussed below, the large rectangles and parallelograms represent the undeformed and deformed shapes of macroscopic devices, respectively, as shown schematically in FIG. 4. The small circles within these geometrical shapes schematically indicate alloy particles. The small squares and parallelograms found within the larger rectangles and parallelograms, schematically indicate the microstructure (crystal lattice) of the alloy. From FIG. 4, the changes in microstructure (crystal lattice) that occur when moving from austenite to martensite to deformed martensite are readily apparent.

It should be noted that for ease of presentation, the microscopic and macroscopic changes resulting from processes 22 and 24 in FIG. 3A and processes 27 and 28 in FIG. 3B have been shown separately. It should be understood that in both Figures the respective pairs may occur isothermally. However, in all circumstances, processes 22 and 24 in FIG. 3A occur below temperature Mₛ, while in FIG. 3B processes 27 and 28 occur between Mₛ and A_{f}.

Medical devices formed from SMAs rely on a shape memory effect (SME) to achieve their desired results. However, the use of the SME in medical applications is attended by two principal disadvantages. Firstly, using the SME requires a device that must be heated inside the human body entailing risk of damage to human tissue. Secondly, use of devices based on the SME does not provide the long-term compression required in many applications.

As mentioned above, many SMAs exhibit superelastic (SE) behavior, characterized by a large nonlinear recoverable strain upon loading and unloading. Referring now to FIG. 5, there is illustrated SE behavior when the device is initially in a stable austenitic state, that is at temperatures above A_{f} but below M_{d}. It should be noted that throughout this text all operations take place at temperatures below M_{d}. The device is deformed 34 so as to cause formation of a metastable martensitic state. This state is represented in FIG. 5 by the region above diagonal line 14 representing the CC relationship. The martensite formed is commonly referred to as stress-induced martensite (SIM). Removal 36 of the distorting force returns the alloy to its austenitic state and the device elastically reverts to substantially its original shape.

In FIG. 5, the large parallelograms indicate a deformed device in a metastable martensitic state, while the rectangles indicate an undeformed device in its austenitic state. The changes in microstructure, i.e. the phase transformation from austenite to deformed martensite in the alloy itself are shown as changes in the small geometrical shapes within the larger parallelograms and rectangles. These changes are schematically illustrated in FIG. 4 discussed above.

For a clearer presentation, processes 34 and 36 are not shown as overlapping. They may, and often do, occur at the same temperature. In all cases the temperature must be above the SMA's A_{f} temperature and the stress must be above CC. Heating 35 may therefore occur as shown in FIG. 5 provided that the temperature remains below Md.

U.S. Pat. No. 4,665,906 dated May 19, 1987, U.S. Pat. No. 5,190,546 dated Mar. 2, 1993, and U.S. Pat. No. 6,306,141 dated Oct. 23, 2001, to Jervis entitled “Medical Devices Incorporating SIM Alloy Elements” as well as U.S. Pat. No. 5,067,957 to Jervis dated Nov. 26, 1991, entitled “Method of Inserting Medical Devices Incorporating SIM Alloy Elements”, disclose a number of medical devices, which use elements formed from a stress-induced martensite alloy. It is disclosed therein that the use of stress-induced martensite (SIM) decreases the temperature sensitivity of the devices, making them easier to position in and remove from the human body.

Carotid angioplasty and stenting are alternatives to surgery for the treatment of atherosclerotic, carotid-artery, and randomized clinical trials. The biocompatibility and shape recoverability of self-expanding SMA stents make them useful for this procedure. Commonly, superelastic behavior is used to insert self-expanding stents. Self-expanding stents are manufactured with a diameter larger than that of the target vessel, crimped to transform austenite to stress-induced martensite, and restrained in a delivery system (catheter), before being elastically released into the target vessel. Recently mesh stents have replaced coil stents. Mesh stents provide some advantages compared with coil stents, but the installation into the restraining catheter is problematic. Using SIM elements requires a technical refinement for their installation, since it requires using special restraining instruments. Mesh stents are discussed in, for example, “An Overview of Stent Design” by T. W. Duering and D. E. Tolomeo published in Proceedings of the International Conference on Shape Memory and Supereleastic Technologies SMST-2000, Ed. S. M. Russell and A. R. Pelton, pp 585-604.

U.S. patent application Ser. No. 09/795,253 filed Feb. 28, 2001 entitled “Staples For Bone Fixation” to the present Applicant, discloses a shape-memory alloy bone staple and associated apparatus for deforming the staple by increasing the span length for insertion thereof into the bone. The deformation range of the staple allows the staple to revert to its shape when the temperature change provides transformation to the austenitic phase.

U.S. patent application Ser. No. 10/237,359 filed Sep. 9, 2002 by the present Applicant entitled “Intratubular Anastomosis Apparatus”, which is incorporated herein by reference, discloses an intratubular anastomosis apparatus for joining organ portions of a hollow organ after intussusception thereof, including an anastomosis ring, and a crimping support element for use therewith. The anastomosis ring includes a length of a wire formed of a shape memory alloy defining a closed generally circular shape, having a central opening, and having overlapping end portions. The anastomosis ring and the shape memory alloy assume a plastic or malleable state at a lower temperature, and an elastic state at a higher temperature. The anastomosis ring thereby retains a preselected configuration at the lower temperature, and an elastic crimping configuration upon reverting to the second, higher temperature.

U.S. application Ser. No. 10/237,505 filed Sep. 9, 2002 by the present Applicant entitled “Intussusception and Anastomosis Apparatus”, which is incorporated herein by reference, discloses an apparatus for intratubular intussusception and anastomosis of a preselected wall portion of a hollow organ. The apparatus includes an anastomosis ring and further includes a length of a wire formed of a shape memory alloy defining a closed generally circular shape, having overlapping end portions. The anastomosis ring assumes a plastic or malleable state when at a lower temperature, and an elastic state when at a higher temperature, thereby enabling the anastomosis ring to retain a preselected configuration at the lower temperature, and an elastic crimping configuration upon reverting to the higher temperature.

U.S. application Ser. No. 10/158,673, entitled “Surgical Clip Applicator Device”, filed May 30, 2002, which is itself a continuation-in-part application of U.S. application Ser. No. 09/592,518, entitled “Surgical Clips”, filed Jun. 12, 2000, by the present Applicant, the contents of both of which are incorporated herein, by reference, discloses an anastomosis clip applicator device for applying a surgical clip. The clip is formed at least partly of a shape memory alloy, to press together adjacent wall portions of adjacent hollow organ portions so as to effect anastomosis therebetween. The applicator device allows for the introduction and application of the surgical clip into adjacent hollow organ portions, such that the surgical clip compresses together the adjacent walls of the hollow organ portions, and thereafter causes the cutting apparatus to perforate the adjacent pressed together organ walls to provide patency through the joined portions of the hollow organ. The clip is formed of a shape memory alloy, which assumes a plastic or malleable state when at a lower temperature, and an elastic state when reaching a higher temperature. The clip retains a preselected configuration at the lower temperature, and an elastic configuration upon reverting to the higher temperature.

Additional prior art using SMAs for medical devices includes: U.S. Pat. No. 3,620,212 to Fannon et al. which discloses an SMA intrauterine contraceptive device, U.S. Pat. No. 3,786,806 to Johnson et al. which discloses an SMA bone plate, and U.S. Pat. No. 3,890,977 to Wilson which discloses an SMA element to bend a catheter or cannula.

U.S. Pat. No. 4,233,690 to Akins dated Nov. 18, 1980 entitled “Prosthetic Device Couplings,” discloses a prosthetic element securely joined to a natural element of the human body using a ductile metal alloy coupling member. The member has a transition-temperature range and can be deformed from its original shape at a temperature below its transition-temperature. Heating the coupling member to a temperature above the transition temperature causes the coupling to try to return to its original shape and effect a secure join.

There are difficulties with prior art SMA-based medical devices and methods for their use.

SMA-based devices which employ the SME require heating, as well as heating the applicators used in positioning the devices. Typically, heating is needed to bring the alloy to a temperature above its A_{f} temperature (see FIGS. 3a and 3B). This heating is cumbersome and at times difficult to achieve, particularly if the device is to be positioned inside the body. Heating may damage sensitive biological tissue. An additional disadvantage of an SMA device based on the SME is that such a device typically does not provide a “recovered” force over extended periods of time, i.e. long-term compression.

SMA devices using the SE effect require relatively substantial loads to generate the desired effect as will be discussed herein below. The applicator of a device based on the SE effect and positioning of the device is generally complicated often rendering surgery difficult if not impossible.

### SUMMARY OF THE INVENTION

The present invention is intended to provide a method for using shape memory alloys (SMA) to provide long-term compression, generally on body tissues. The method allows for the use of low loads and the loads are applied at temperatures at which the SMA is at least partially in its martensitic phase.

The present invention is intended to provide a method for using SMAs which allows for greater shape restoration then prior art methods.

The present invention is also intended to provide a method for using SMAs having Af temperatures below body temperature.

The present invention is further intended to provide a method for using SMAs in medical devices where restraining of the device is effected by body tissue.

The present invention is also intended to provide a method for using SMAs which allows for greater recovery of the applied distorting force.

The present invention is also intended to provide a method for using devices containing SMAs which allows for easier positioning when using a device applicator.

The present invention is also intended to provide medical devices formed from SMAs, employing stress-retained martensite and employing the superelastic plasticity (SEP) effect.

There is provided according to one aspect of the present invention a method for utilizing a deformable article of manufacture adapted to have selectable first and second predetermined configurations and being formed at least partly of a shape memory alloy. The method includes the steps of: deforming the article under a deforming force from the first predetermined configuration to the second predetermined configuration while the shape memory alloy is, at least partially, in its stable martensitic state and at a first temperature; applying a resisting force to the deformed article of manufacture using a restraining means; heating the article from the first temperature to a second temperature in the presence of the resisting force, thereby transforming the alloy from its stable martensitic state to its metastable stress-retained martensitic state, while the article remains in its second configuration; and removing the resisting force thereby allowing the alloy to transform to its austenitic state and the shape of the article to be restored substantially to the first configuration.

In a preferred embodiment of the method of the present invention, the article of manufacture is a medical device.

In another embodiment of the method, the method further includes the step of positioning the deformed article within the human body while the deformed article is restrained by the restraining means. In some instances of this embodiment, the step of heating is a step of automatically warming to body temperature when the article is positioned in or near the human body, body temperature being above the alloy's A_{f} temperature.

In yet another embodiment of the method, the method further includes the step of positioning the deformed article within the human body. In this embodiment, the restraining means is body tissue. In some instances of this embodiment, the step of heating is a step of automatically warming to body temperature when the article is positioned in or near the human body, body temperature being above the alloy's A_{f} temperature.

In another embodiment of the method, the method further includes the step of cooling prior to the step of deforming, and the step of cooling includes cooling the article to the first temperature such that the shape memory alloy transforms, at least partially, into its stable martensitic state. In some instances of this embodiment, the step of cooling includes cooling the article from the alloy's austenitic state to a state wherein the alloy is at least partially in its stable martensitic state.

In another embodiment of the method, the step of heating includes heating the article until A_{f}, that the shape memory alloy preserves its stable martensitic state.

In yet another embodiment of the method, the step of heating is a step of automatically warming to body temperature when the article is positioned in or near the human body, body temperature being above the alloy's A_{f} temperature.

In still another embodiment of the method the step of heating includes the step of heating to above the alloy's A_{f} temperature. In still another embodiment the first temperature is below Mₛ. In yet another embodiment, the first temperature is below Mₛ and the second temperature is above A_{f}. In another embodiment, the first temperature is below A_{f} and the second temperature is above A_{f}.

In another embodiment of the method, the step of removing is effected isothermally.

In an embodiment of the method, the restraining means in the step of applying is body tissue

In another embodiment of the method of the present invention, a deformation is effected in the step of deforming by a means for deforming which is the same means as the restraining means in the step of applying. The resisting force in the step of applying is substantially a continuation of the deforming force provided in the step of deforming employed to deform the article.

In an embodiment of the method, the step of deforming includes a deformation effected by a means for deforming which is the same means as the restraining means in the step of applying. In some instances of this embodiment, the restraining means in the step of applying is body tissue.

In another aspect of the invention there is provided a selectably deformable article of manufacture. The article is adapted to have selectable first and second predetermined configurations, the article being formed at least partly of a shape memory alloy. The shape memory alloy is at least partially in its stable martensitic state and at a first temperature, thereby facilitating deformation of the article from the first predetermined configuration to the second predetermined configuration. The shape memory alloy is further transformable from the stable martensitic state to a metastable stress-retained martensitic state, when heated to at least a second temperature in the presence of a predetermined resisting force. The resisting force impedes transformation of the shape memory alloy from the metastable stress-retained martensitic state to an austenitic state and thereby also impedes reversion of the article of manufacture from the second predetermined configuration to the first predetermined configuration.

In an embodiment of the article, the first temperature is below Mₛ. In another embodiment, the first temperature is below A_{f}. In yet another embodiment of the article the second temperature is above A_{f}. In a further embodiment of the article, the second temperature is lower than normal body temperature. In yet another embodiment of the article of manufacture, the stable martensitic state is attained by cooling the alloy to a first temperature below its Mₛ temperature from above its A_{f} temperature.

In another embodiment of the article, the metastable stress-related martensite transforms to the austenitic state upon removal of the resisting force and the article reverts to its first configuration from its second configuration.

In still another embodiment of the article, the article of manufacture is a medical device. Often when a medial device is used the second temperature is substantially body temperature and A_{f} is below body temperature.

In other embodiments of the article, the medical device may be a surgical clip, an anastomossis ring for crimping adjacent intussuscepted organ wall portions against a generally tubular crimping support element, a staple for bone fixation, an expandable bone fastener, an expandable bone anchor, a coil or mesh stent for disposing in a human vessel so as to provide improved liquid circulation therethrough, an intrauterine device, a heart valve retaining ring, a clamp device for securing tissue, and a blood vessel filter.

In some embodiments of the deformable article of manufacture, the second temperature is lower than body temperature and A_{f} is below the second temperature. In still other embodiments of the deformable article of manufacture, the second temperature is body temperature, body temperature being above the alloy's A_{f} temperature.

### BRIEF DESCRIPTION OF THE DRAWINGS

The present invention will be more fully understood and its features and advantages will become apparent to those skilled in the art by reference to the ensuing description, taken in conjunction with the accompanying drawings, in which:

FIG. 1 illustrates the martensite/austenite phase transformations as a function of temperature for a shape memory alloy (PRIOR ART);

FIG. 2 is a schematic representation of the phases (states) in a shape memory alloy subjected to controlled stress and temperature changes;

FIGS. 3A and 3B are schematic representations illustrating the shape memory effect of a device subjected to controlled stress and temperature changes (PRIOR ART);

FIG. 4 is a schematic representation illustrating the different microstructures possible in shape memory alloys and the macroscopic changes of a device made from such alloys resulting from such changes in microstructure;

FIG. 5 is a schematic representation illustrating the superelasticity effect of a device subjected to controlled stress changes (PRIOR ART);

FIG. 6 is a schematic representation illustrating the superelastic plasticity (SEP) effect of a device subjected to controlled stress and temperature changes;

FIG. 7 is a schematic representation illustrating the phase transformations between austenite and stress-induced martensite (SIM) subjected to controlled stress and temperature changes (A_{f}>body temperature) (PRIOR ART);

FIG. 8 is a graphical representation of force versus closing distance in shape memory alloy staples based on SIM (A_{f}>37° C.) and SRM (A_{f}<37° C.);

FIG. 9 is a graphical representation of comparative loading force versus extension applied to shape memory alloy staples based on SRM (A_{f}<37° C.) and SIM (A_{f}<37° C.);

FIG. 10 is a schematic illustration of a surgical clip and cross-sectional views thereof;

FIG. 11 is a schematic illustration of an anastomosis ring and cross-sectional views thereof;

FIG. 12 is a schematic illustration of an anastomosis ring in crimping engagement against a crimping support element;

FIG. 13 is a schematic cross-sectional view taken from FIG. 12 indicating an anastomosis ring in crimping engagement with an intussuscepted hollow organ portion;

FIG. 14 is a schematic perspective view of a closed bone staple;

FIG. 15 is a schematic perspective view of an open bone staple;

FIG. 16 is a schematic perspective view of an open bone staple applied to a fractured bone;

FIGS. 17A and 17B are schematic views of a bone anchor in its closed and open positions respectively;

FIG. 18 is a schematic view of an expandable bone fastener;

FIG. 19 is a schematic perspective view of the bone fastener in FIG. 18;

FIG. 20 is a schematic perspective view of the bone fastener in FIG. 18 with closed anchoring projections;

FIG. 21 is a schematic view of a coil stent;

FIG. 22 is a schematic view of a vessel filter prior to final installation;

FIG. 23 is a schematic view of the vessel filter of FIG. 22 after installation;

FIG. 24 is a schematic perspective view of a clamp in an open configuration;

FIG. 25 is a schematic perspective view of the clamp of FIG. 24 in a closed configuration;

FIGS. 26A and 26B area schematic views of a dental implant before and after implantation respectively; and

FIG. 27 is a schematic view of a retaining ring for use with an artificial heart valve.

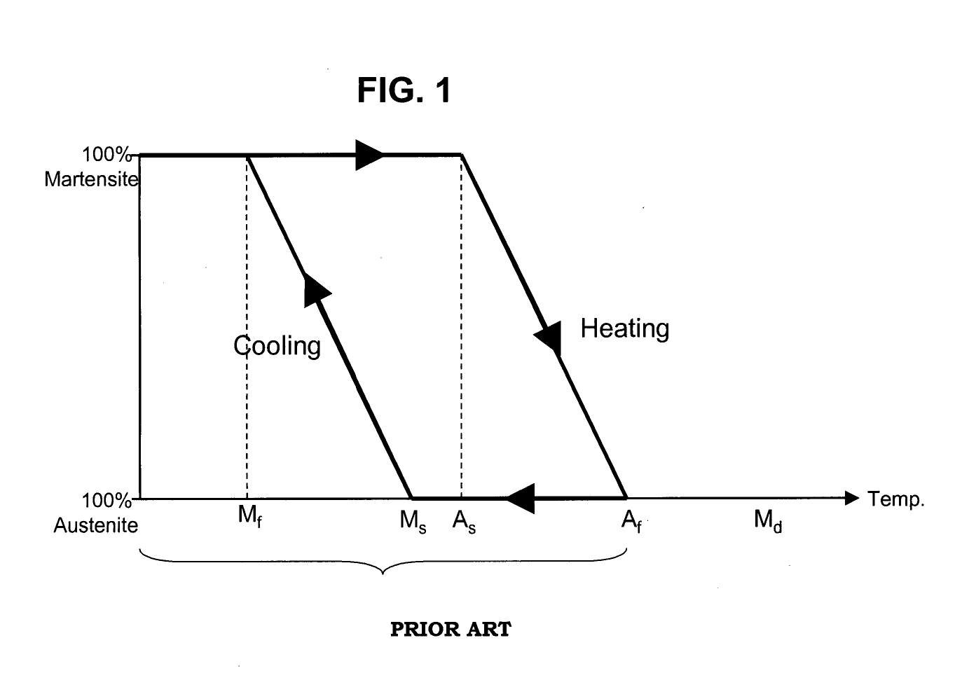

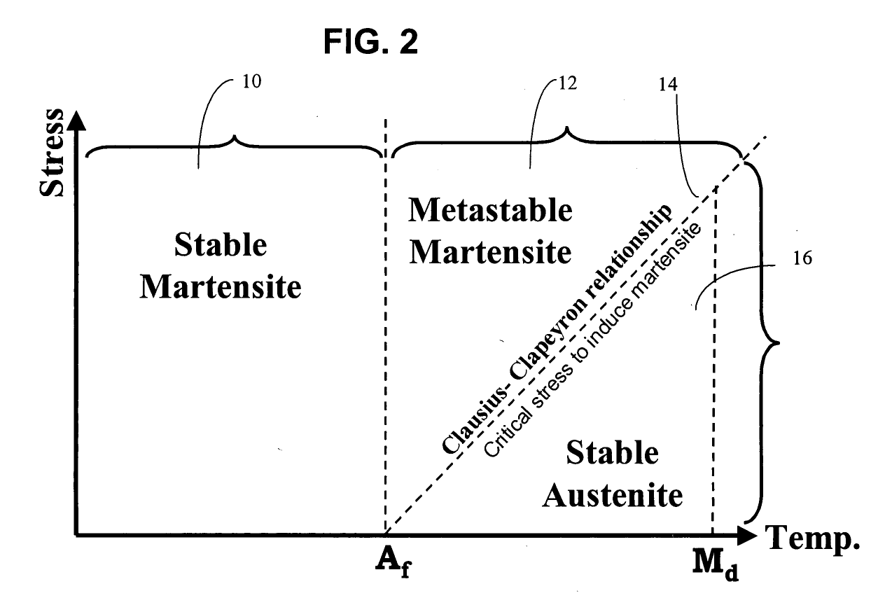

### DETAILED DESCRIPTION OF THE INVENTION

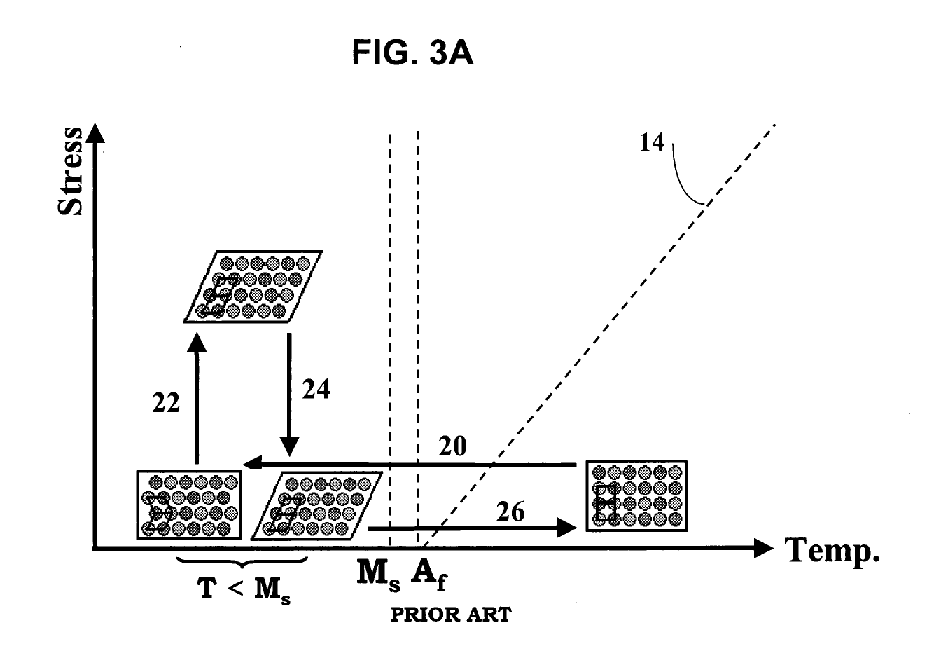

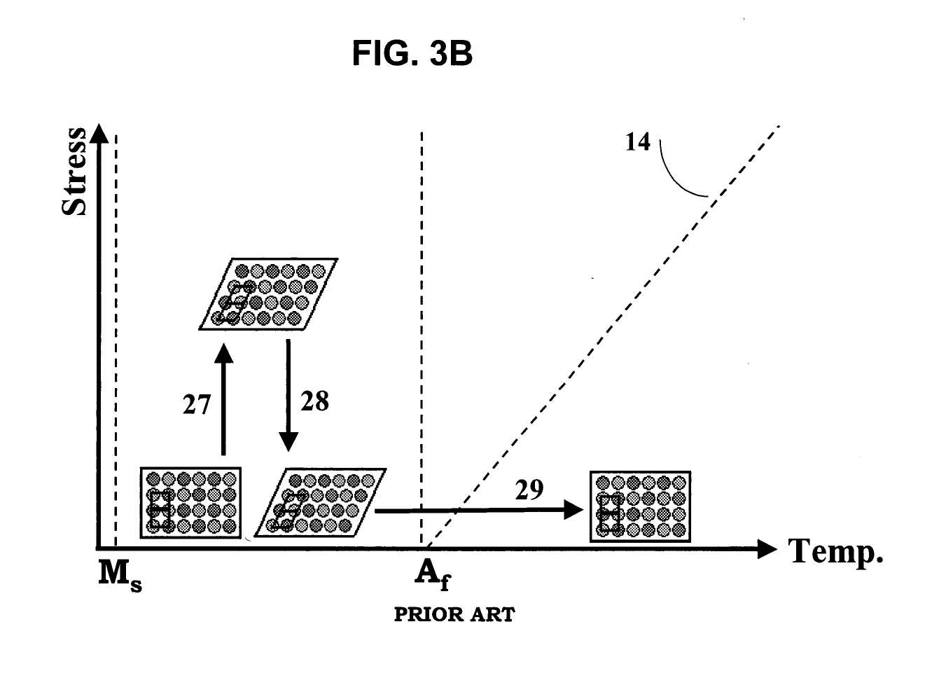

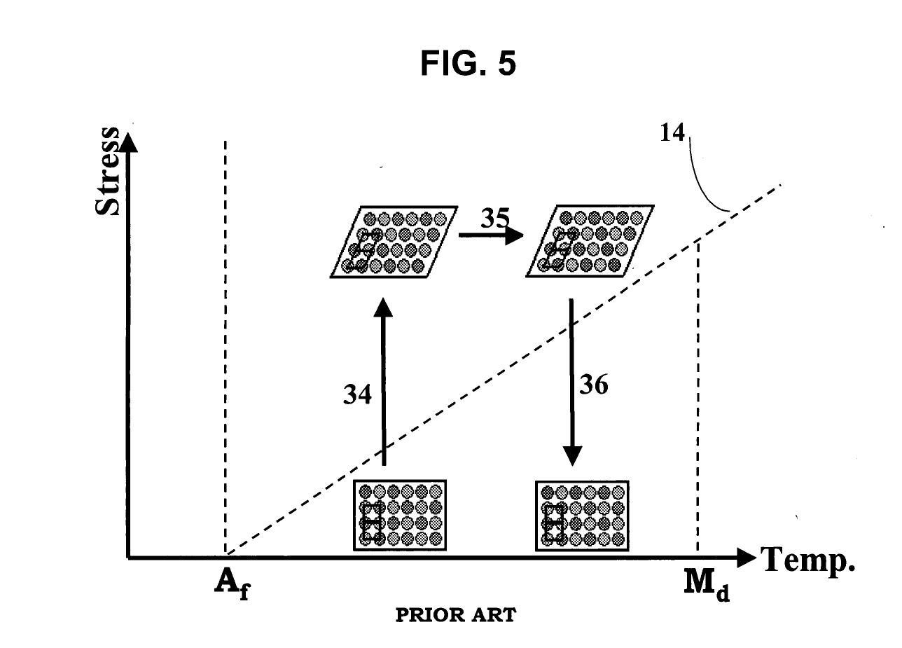

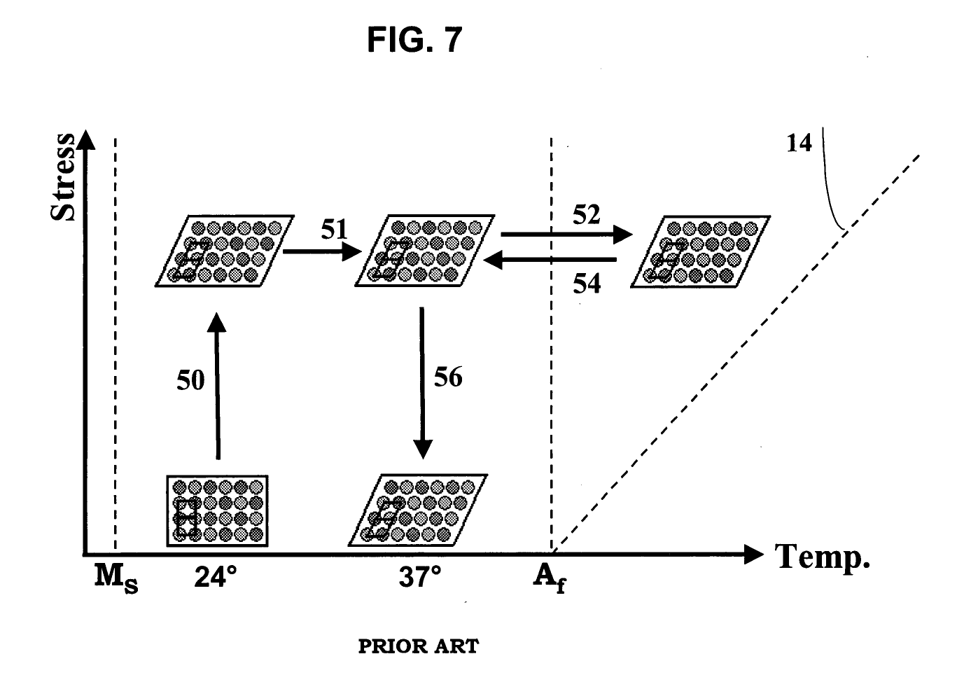

The present invention inter alia teaches a method for using a device, typically a medical device, formed, at least in part, from a shape memory alloy. The method makes use of an effect referred to herein as the superelastic plasticity (SEP) effect. The operative phase responsible for this effect is herein referred to as stress-retained martensite (SRM). As will be clear from the discussion below, using the SEP effect based on SRM in medical devices, for example, has distinct advantages over devices using solely the SME (FIGS. 3A and 3B discussed above and FIG. 7 discussed below) and the SE effect (FIG. 5). The SEP effect (and SRM on which it is based), medical devices using this effect, and a method for using SMA devices employing this effect are the basis of the invention described below.

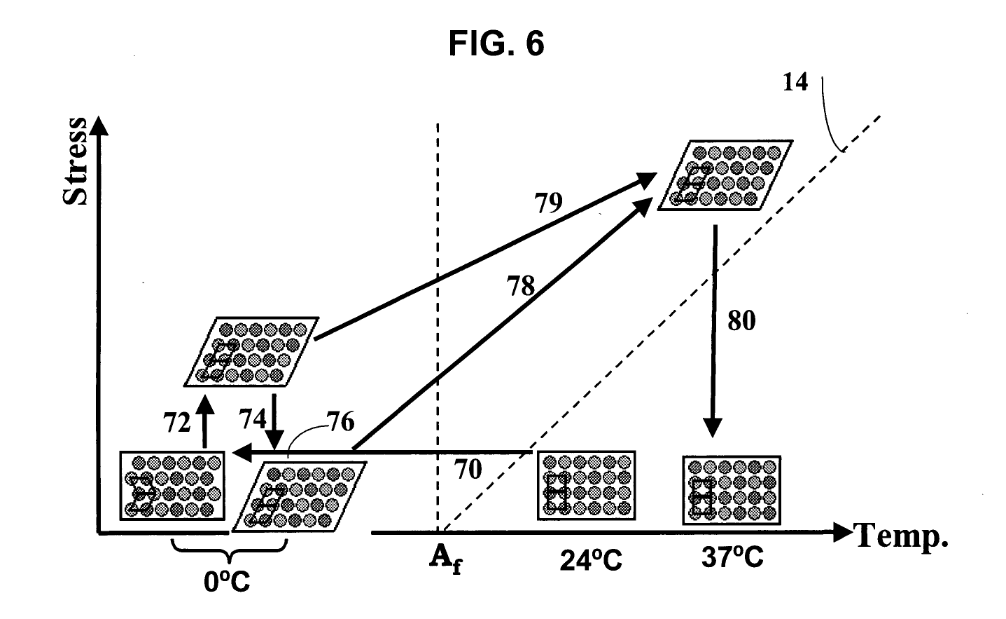

Reference is now made to FIG. 6, where the superelastic plasticity (SEP) effect relating to, for example, bone staples, bone anchors, expandable bone fasteners, stents, or anastomosis clips and the like, is schematically illustrated. The SEP effect used in the method of the present invention represents an SMA's transformation from at least a partially martensitic phase to its austenitic phase via its metastable martensitic phase.

FIG. 6 schematically illustrates the steps in applying the SEP effect to a device formed from an SMA having SRM properties. The steps illustrated are as follows.
1. Cooling 70 a device from the SMA's austenitic state to a temperature where the SMA is at least partially martensitic. During this step the device retains its original shape. The starting and ending temperatures for cooling 70 shown in the Figure are typical non-limiting values. However the lower temperature must be below A_{f};
2. Deforming 72 the device from its original shape by applying a load, thereby producing deformed martensite;
3. Removing 74 the load while the device retains its deformed macroscopic shape and while the SMA retains its deformed martensite micro-structure;
4. Restraining 76 the device so that it retains its deformed shape;
5. Heating 78 the restrained device to a temperature in excess of A_{f}, thereby causing a transformation from the alloy's deformed martensitic state to a stress-retained martensitic state. The stress-retained martensitic state is represented by the region of the graph above diagonal CC line 14. Typically, but without being limiting, this heating may be effected by warming to body temperature (37° C.);
6. Removing 80 the restraint so that the device returns to its original shape with the alloy reverting to its austenitic state.

It should be noted that operations 74 and 76 may be achieved differently for different mechanical devices. For example, a stent is cooled 70, deformed 72, and the deforming load removed 74. The stent is then disposed 76 in its deformed shape into a suitable instrument, such as a catheter, where it is restrained 76 and allowed to warm 78. In some medical devices, such as an anastomosis surgical clip, the medical device is cooled 70 to a martensitic state, disposed and deformed (opened) 72 by an applicator device. As the clip warms 79 directly to ambient temperature, the clip is restrained 76 in its open, deformed configuration by the same applicator device. In the case of the stent, two different devices are used, one for deforming the stent by applying 72 the original load and another, the catheter, for restraining 76 the SMA device during warming. In the surgical clip case, a single device may be used, first to apply 72 a load to deform the clip and then to restrain the device when it is heated 79. Accordingly, removing step 74 may or may not be required depending on the device used.

In other embodiments, human tissue may serve as the restraining means. For example, when SMA bone staples are used, fractured bone tissue acts as the restraining device during the warming process. As seen in FIG. 6, a staple is cooled 70, deformed 72, and the deforming load removed 74. The staple is then disposed using a cooled pincer into special holes in the bone tissue where it is restrained and allowed to warm 78. No external shape restraining applicator or device is required. Bone tissue restrains the staple in its deformed SRM state. Gradually, the staple's legs cut into the bone tissue, and the staple returns to its original shape with the SMA from which the staple is formed transforming 80 from SRM to austenite.

The step of removing 80 discussed may be done gradually and may not include the removal of the entire resisting force. For example, in stents the venous tissue may continue to apply a small resisting force which will prevent the stent from completely recovering its original shape. Bone staples gradually return to substantially their initial shape as osteosynthesis proceeds. For the examples given, the step of removing 80 is a physiological change resulting in a decrease in the load without its complete removal. In other devices, such as the surgical clip and the filter discussed below, there is a removal of an actual restraining means.

It should readily be understood that the step of cooling 70 is optional; there may be instances wherein the SMA of the device is already in a partially martensitic state and the step of cooling 70 is unnecessary.

In order to better understand the advantages of the present invention to be discussed further below, another stress-induced martensite (SIM) process is presented in FIG. 7 to which reference is now made. FIG. 7 schematically shows a prior art, temperature-manipulated, SIM transition at temperatures below A_{f} but above Mₛ. Application of a deforming stress 50 to an SMA in a 100% austenitic state (rectangle in FIG. 7) produces a deformed macrosopic device (large parallelograms in the upper row) and a stress-induced martensitic microstructure (small parallelograms within the large parallelograms). Heating 51 and 52 from within the temperature range Mₛ to A_{f} to a temperature above A_{f} results in the formation of metastable martensite. Generally, in typical prior art SIM transformations, the alloy has an A_{f}>body temperature (BT). It should be remembered that stable austenite is only present in the region below diagonal CC line 14.

On cooling 54 to body temperature (37° C.), the device remains deformed and the alloy exists in a stable deformed martensitic state (middle parallelogram, upper row). The device, typically a medical device such as a bone staple, does not revert fully to its original shape. The device (bottom parallelogram) also remains somewhat deformed after removal 56 of the deforming stress, and only an incomplete recovery of the applied deforming force is obtained. After removal 56 of the deforming stress, the SMA continues to have a deformed martensitic microstructure.

FIG. 7 illustrates use of SIM but the Figure indicates that there is little shape restoration. In effect, therefore, if A_{f}>body temperature (BT), the desired work of a SIM-based SMA device can not be attained since substantially complete shape restoration can not be obtained.

It should be noted that the temperatures shown in FIG. 7 are typical, but non-limiting, working temperatures in prior art medical devices using SMAs. The main point in the Figure is that typical prior art uses alloys where BT<A_{f}.

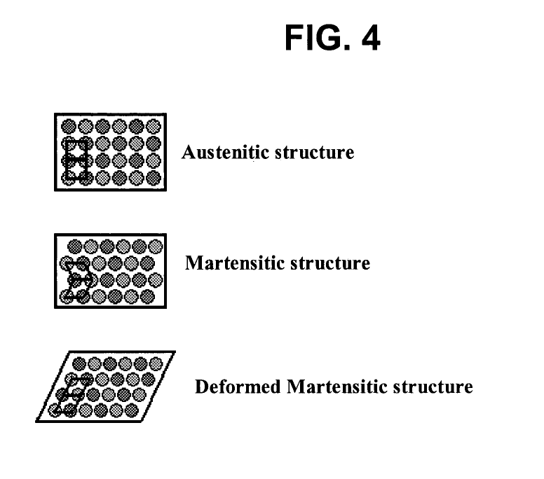

FIG. 7 should be viewed in conjunction with FIG. 4 where the macroscopic condition of the device and the microstructure of the alloy are illustrated.

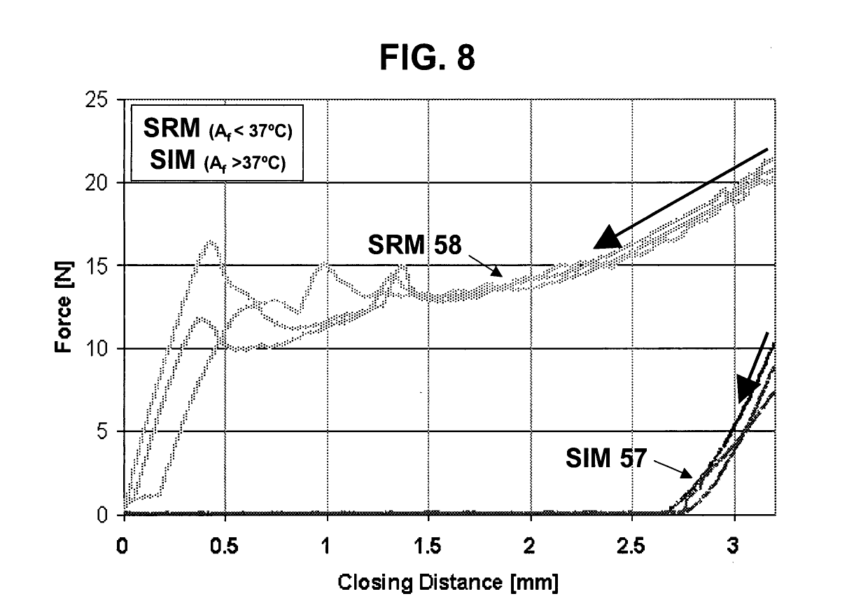

FIGS. 8 and 9 represent an experimental comparison between bone staples using the SEP effect based on SRM and the SME and the SE effect based on SIM. Inter alia they reveal advantages of SRM over SIM. The tests described below were performed using a force tester equipped with a monitored temperature cell for cooling and heating.

FIG. 8, to which reference is now made, shows a comparison of available force versus closing distance between bone staples made from shape memory alloys having SIM and SRM properties. The results discussed in relation to FIG. 8 are equally applicable to other types of devices made from SMAs using these properties.

In FIG. 8, SMA staple 57 using SIM properties underwent stress and temperature changes similar to those shown in FIG. 7. The SMA had an A_{f}>body temperature. The SMA staple 58 using SRM properties underwent stress and temperature changes similar to those shown and discussed in conjunction with FIG. 6. The SMA in staple 58 had an A_{f}<body temperature.

Curve associated with staple 57 indicates the recovered force available from a bone staple constructed from an SMA having an A_{f} temperature (42° C.) higher than body temperature (37° C.). The staple was stretched to 3.5 mm at 20° C., heated to 45-50° C. and then cooled to about body temperature. As the closing distance was reduced, that is, as the distance between the test machine's grippers was reduced, recovery of the staple's original shape was incomplete. The recovery was only about 0.5 mm.

Curve associated with staple 58 indicates the recovered force available from a bone staple constructed from an SMA having an A_{f} temperature (20° C.) lower than body temperature (37° C.). The staple was stretched to the same 3.5 mm at 0° C. and heated directly to 37° C. As the distance between the test machine's grippers was reduced, the reversion of the staple to its original shape was substantially complete. Almost the entire 3.5 mm was recovered. Moreover, the maximum value of the “recovered” force for the SRM staples was about twice the maximum force “recovered” from the SIM staples.

These are significant differences which have important implications for the healing of fractured bones. Despite existing opinion, currently used SIM staples with A_{f}>body temperature apply practically no compression on the fracture line since their force is very quickly reduced. However, the compression force of SRM staples is maintained almost throughout their entire closing distance. These results show that only SRM staples can assure long-term compression osteosynthesis

Referring now to FIG. 9, there is seen a graphical representation of comparative loading/unloading versus extension when applied to two staples (A_{f}=20° C.<body temperature). One of the staples employed the SEP effect based on SRM while the second staple used the SE effect based on SIM. The SE effect is effectively the same as that shown in FIG. 5 while the SEP effect based on SRM is effectively the same as that shown in FIG. 6. The staples were mounted on a force tester and gradually opened to a distance of 2.5 mm at different temperatures. Gradually, the grippers of the force tester were brought closer together, allowing the staple to close.

A load of up to 60 N was needed to open the staple using SIM properties at a temperature of 24° C. (curve 60). The temperature was increased to body temperature (37° C.) (69) and the “recovered” load, the result of the transformation from SIM to austenite, is shown in curve 62. This is the SE effect.

By comparison, the required load to deform and open the staple using SRM properties at 0° C. was about 26 N (curve 64). The temperature was increased to body temperature (37° C.) (curve 66). When the temperature approached A_{f}, stress-retained martensite (SRM) was formed and retained up to 37° C. Load recovery occurred with the transformation of SRM to austenite (curve 68). This is the SEP effect.

Recovery curves 62 and 68 for SIM and SRM devices respectively are very similar. However, the respective applied loads, curves 60 and 64, are different with the load required to deform SIM being about 2.5 times greater than that required to deform SRM. This feature represents a substantial advantage for the use of SRM instead of SIM in devices, such as bone staples, clips and stents and other similar devices. It is also clear from the Figure that a much larger part of the applied load is “recovered” with SRM staples. Another advantage, not readily recognizable from the Figure, is that in the case of bone staples and other similar devices, SRM does not require a special shape-retaining instrument when applying the device to the body site. Body tissue can be used as the shape-retaining “instrument”.

To summarize, the SEP effect must occur with an SMA in at least a partial martensitic state. A_{f} is set below the working temperature in SMA-based devices using the SEP effect. Typically, A_{f} is set below body temperature when an SRM-based medical device is employed. Generally, SEP shape restoration does not require external heating in SRM-based medical devices since the body typically serves as the heat source. After heating, shape is restored by load removal, typically, but not necessarily, at isothermal conditions. The SEP effect enables substantially complete recovery of the device's original shape, thus providing long-term compression on body tissues. The SEP effect generally allows for the recovery of more of the applied load than the SE effect while the initial deforming load for the former is significantly less than the latter. Additionally, the SEP effect can often be effected in medical devices without using special restraining devices. Body tissue, such as bone, may be used as the restraining means. These advantages are of great practical importance.

Use of SRM in Medical Devices

There follows below examples of medical devices which are preferably formed, at least partially, of a shape memory alloy (SMA). The SMA uses the SEP effect based on SRM, typically at body temperature. However, body temperature should be viewed only as an exemplary temperature and should not be considered limiting.

Surgical Anastomosis Clips

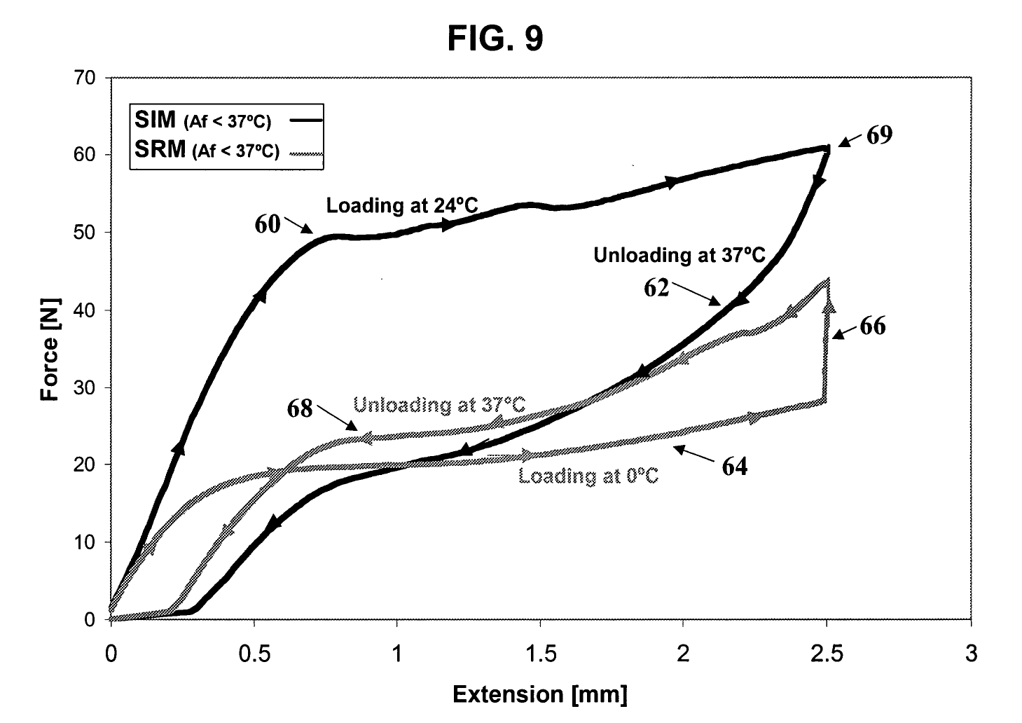

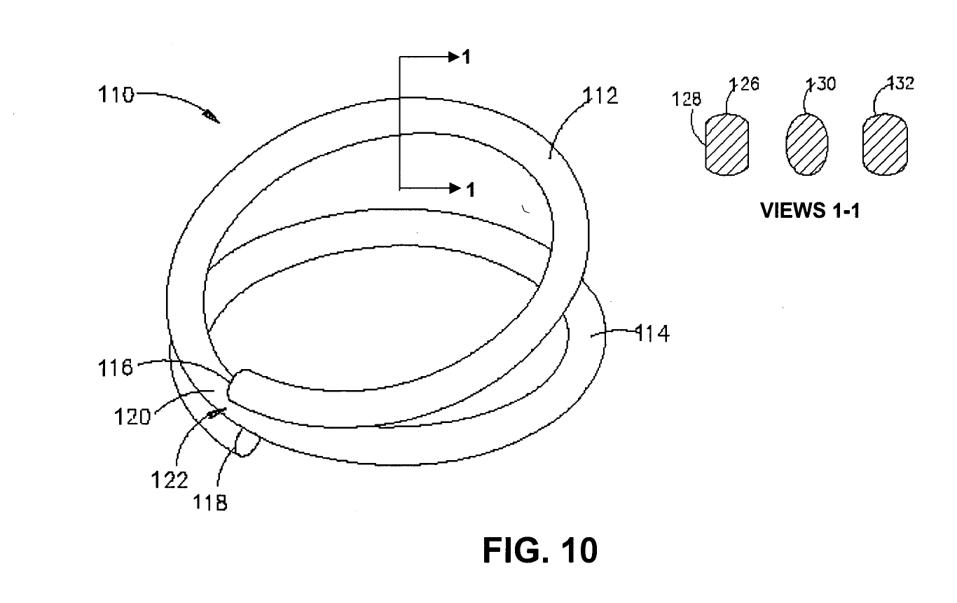

Referring now to FIG. 10 in accordance with an embodiment of the present invention, there is seen a surgical clip, generally referenced 110, illustrated in an open configuration. Clip 110 is typically wire-like, formed at least partly of a shape memory alloy, and is of a coiled configuration so as to include a pair of loops referenced 112 and 114, having respective ends referenced 116 and 118. Each of loops 112 and 114 defines a complete circle from its end to a point referenced 120 midway along the coil. Thus, coil 110 defines two complete circles from end 116 of loop 112 to end 118 of loop 114.

While the various embodiments of clip 110 of the present invention are illustrated as defining circular shapes, it will be appreciated by persons skilled in the art that the present invention may, alternatively, define any closed geometric shape, such as for example, an ellipse. Surgical clips formed having other configurations are used where surgically appropriate, in accordance with the organ size, position and other factors.

While the entire clip 110 may be formed of a shape memory alloy, it is essential that at least an intermediate portion generally referenced 122 of clip 110 is formed of a shape memory alloy displaying SRM behavior. When the clip is mounted on an applicator device and cooled to or below a predetermined first temperature, clip 110 transforms to a plastic martensitic state. Loops 112 and 114 may be moved apart by the applicator as seen in FIG. 10. When heated to or above a second temperature, which is typically below body temperature, and while a resistance force is applied by the applicator so as to keep loops 112 and 114 in a spaced-apart configuration, the stressed shape memory alloy transforms to a metastable stress-retained martensitic state. When clip 110 is removed from the applicator and applied to adjacent walls of a pair of juxtaposed hollow organ portions (not shown), so as to cause anastomosis therebetween, the tissue of the adjacent walls provide a resistance force to a compressive force exerted on loops 112 and 114 by the shape memory alloy of intermediate portion 122. Consequently the stressed shape memory alloy transforms from its stress-retained martensitic state to its austenitic state. The shape of the clip is restored thereby providing for compressive anastomosis.

In order to further control the pressure applied to the tissue walls at the point of contact with clip 110, the cross-section of the wire forming the clip may be varied, both in cross-sectional area and in shape. Referring now to cross-sectional views 1-1 in FIG. 10, there are seen cross-sectional views of alternative profiles taken along line 1-1 of surgical clip 110. There is seen a generally circular cross-sectional profile referenced 126, having planar surfaces referenced 128 formed therein according to an alternative embodiment of the present invention, an elliptical profile referenced 130, and an elliptical-type profile referenced 132.

In accordance with a preferred embodiment of the invention, suitable surgical clips and an applicator device for applying such clips are disclosed in Applicant's co-pending U.S. application Ser. No. 10/158,673, entitled “Surgical Clip Applicator Device”, filed May 30, 2002, which is itself a continuation-in-part application of U.S. application Ser. No. 09/592,518, entitled “Surgical Clips”, filed Jun. 12, 2000. Both applications are incorporated herein by reference.

Anastomosis Ring and Crimping Support Element

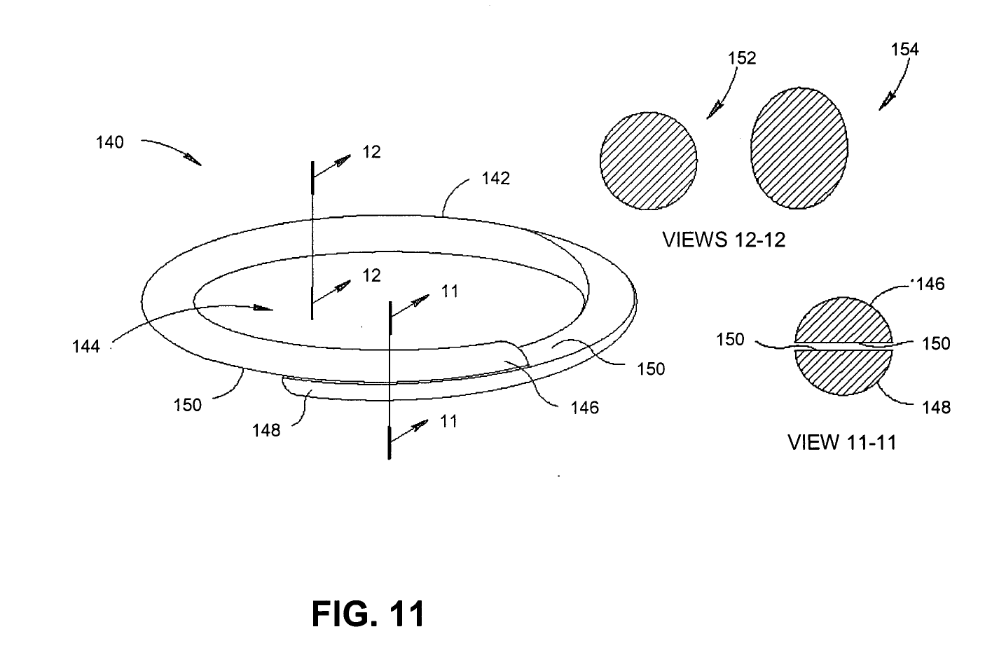

With reference to FIGS. 11, 12 and 13, in accordance with another embodiment of the present invention, there is seen, in FIG. 11 an anastomosis ring generally referenced 140, which is configured from a length of shape memory alloy wire 142 as a closed generally circular shaped ring, having a central opening referenced 144, a predetermined wire thickness and overlapping end portions referenced 146 and 148.

In FIG. 11 there is also seen a cross-sectional view of overlapping end portions 146 and 148 of anastomosis ring 140 as taken along line 11-11. Each of end portions 146 and 148 has a flat contact surface referenced 150 formed thereon so as to provide a similar cross-sectional profile at overlapping portions 146 and 148 as wire 142.

In order to control the pressure on the tissue walls at the point of contact with anastomosis ring 140, the cross-section of the wire forming ring 140 may be varied, in accordance with alternative embodiments of the present invention. In FIG. 11 there are further seen cross-sectional views, which are non-limiting examples only, of alternative profiles taken along line 12-12 of surgical clip 140. There is seen a generally circular cross-sectional profile referenced 152. According to an alternative embodiment of the present invention, there is seen an elliptical profile referenced 154.

When cooled to or below a first temperature, the shape memory alloy of anastomosis ring 140 assumes a stable plastically malleable martensitic state, and an elastic austenitic state, when warmed to or above a second, higher temperature. This stable martensitic state facilitates that anastomosis ring 140 is expanded and retains an expanded configuration at the first, lower temperature. Once ring 140 is warmed to, or above, the second temperature, without the imposition of a resisting force, ring 140 returns substantially to the original configuration.

However, imposing a resisting force thereto by a resistance means, so as to resist clip 140 reverting to its original configuration and thereby to cause ring 140 to exert a compressive force counter to the resisting force, the shape memory alloy assumes a metastable stress-retained martensitic state, so as to apply a predetermined stressing force to the resistance means.

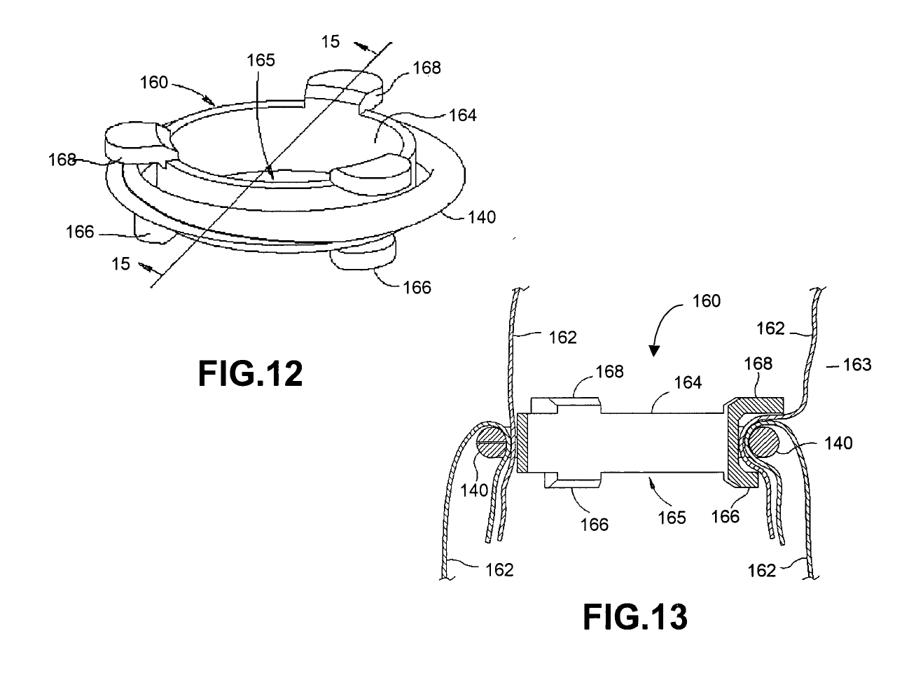

Referring now to FIGS. 12 and 13, there is seen, respectively, a perspective and a cross-sectional view of anastomosis ring generally referenced 140 in crimping engagement with a crimping support element referenced generally 160, in accordance with an embodiment of the present invention. The cross-sectional view seen in FIG. 13 is taken along line 15-15 in FIG. 12. The Figure also shows intussuscepted adjacent walls 162 of organ portion 163. Crimping support element 160 includes a short tubular section referenced 164 with an opening referenced 165 therethrough, proximal and distal end lugs referenced 166 and 168 respectively. An anastomosis ring 140 is cooled to a reduced temperature, below body temperature, where the shape memory alloy transforms from its austenitic to its martensitic state. Ring 140 is easily deformed to an insertable size, so as to fit onto a cooled restraining means of an anastomosis apparatus (not shown). By warming to or above a second temperature, anastomosis ring 140 attempts to revert to its original configuration. As a result of the warming process while the ring's shape is restrained, the shape memory alloy is transformed into its stress-retained martensitic state. When ring 140 is liberated from a restraining means (not shown) it applies a predetermined stressing force to adjacent walls 162 of organ portion 163 and crimping support element 160 as the alloy attempts to revert to its austenitic state, thereby causing anastomosis between adjacent walls 162.

Bone Staples

Clinical experience illustrates that the use of bone staples constructed of a shape memory alloy provides definite advantages in the surgical repair of fractured bones, particularly of small bones, such in maxilla facial, foot and hand surgery.

SMAs having SIM properties have been proposed for this application. However, they have the following problems.

1. If the SMA has a temperature A_{f} above body temperature, the alloy exhibits SME behavior (FIGS. 3A and 3B) and a device may be implanted in a martensitic state. Brief heating will be required to transform the alloy to a metastable martensitic phase, and on re-cooling to body temperature, the metastable martensite returns to a stable martensite state. However, the alloy does not provide complete shape restoration and the compression force is very much reduced. FIG. 8, as discussed above, shows the reduced shape recovery.

2. If the alloy has an A_{f} temperature below body temperature, the alloy exhibits SE (SIM), and the force needed to deform a bone staple is substantially greater than the force applied by the staple to a bone fracture when the staple's shape is restored. This was discussed in conjunction with FIG. 9 above.

3. When the alloy is in an austenitic state, a special instrument is required to deform bone staples and to mechanically conserve the deformed shape. Such an instrument generally prevents easy installation of the staple.

According to embodiments of the present invention, if an SRM alloy is utilized, these disadvantages are substantially overcome. Firstly, SRM utilization provides almost full shape restoration in the presence of a permanent compression force referenced 58 in FIG. 8. Secondly, the force necessary for shape deformation of a staple in a stable martensitic phase is much smaller than when the alloy is in an austenitic state, as discussed above in conjunction with FIG. 9.

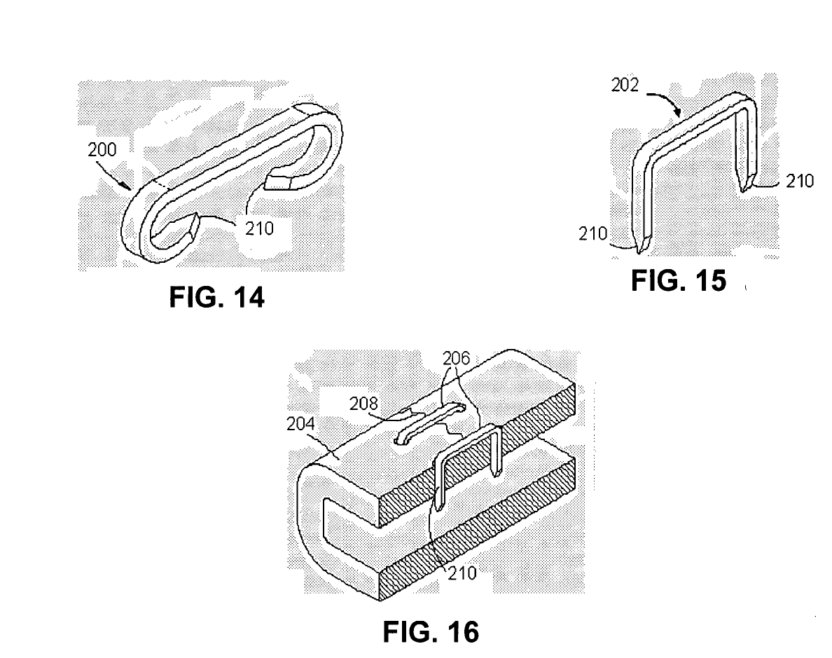

Referring now to FIGS. 14, 15 and 16, in accordance with an embodiment of the present invention, there are seen respectively the closed, open and inserted configurations of a bone staple. In FIG. 14, bone staple, referenced 200, is shown in its closed configuration. After cooling, the SMA is transformed into a stable martensitic state, and the staple is relatively easily deformed to the open configuration referenced 202 shown in FIG. 15. After implantation in a fractured bone 204 as in FIG. 16, staples 206, although naturally warmed to body temperature, remain in a martensitic state. However, the alloy is no longer in a stable martensite state, but has been transformed into a stress-retained martensite state. As the resistance of bone 204 prevents shape restoration, staples 206 attempt to revert to their closed configuration 200, providing a predetermined stressing force to the fracture site 208 as the alloy attempts to revert to its austenitic state.

The physiological process of fracture consolidation takes at least two weeks. In order to relieve the compression on the bone fracture site 208 caused by the SRM state of bone staples 206, a reconstruction of bone cells takes place at fracture site 208. There is a perception that end portion legs referenced 210 of staples 206 are transformed to a closed configuration 200 by apparently “cutting” through the bone 204. During the shape restoration of staples 206, the transformation of SRM to austenite provides an almost constant stress at the fracture site.

Bone Anchor

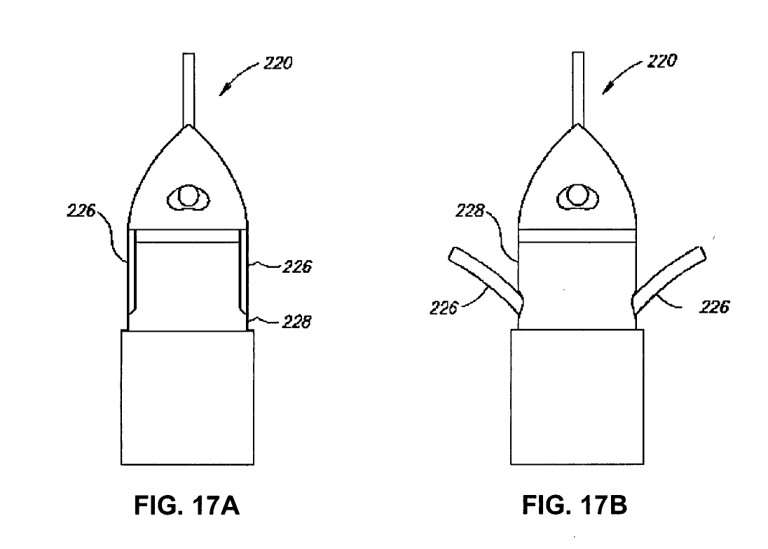

Referring now to FIGS. 17A and 17B, in accordance with an added embodiment of the present invention, the mechanism for utilizing a bone anchor generally referenced 220 is substantially similar to that required for bone staples, as disclosed herein above in conjunction with FIGS. 14, 15 and 16. In preparation for locating bone anchor 220, a hole (not shown) is drilled into a bone. Bone anchor 220 is pre-cooled so that the shape memory alloy of anchor arms referenced 226 is transformed into its stable martensitic state. As indicated in FIG. 17A, arms 226 are deformed against a fastener body referenced 228. Thereafter bone anchor 220 is positioned in the hole in the bone. Body heat warms fastener 220 to body temperature, causing arms 226 to deflect outwards as shown in FIG. 17B against the inner surface of the hole, which provides a resisting force thereto. As a result of warming, the alloy of arms 226 is transformed into its SRM state. The stress-retained martensite attempts to transform into austenite, thereby to cause anchor 220 to be anchored into the bone. Using SRM allows ease of deformation without the need for restraining arms 226 in a closed position but using a special restraining placement device in some cases may be useful.

Expandable Bone Fastener

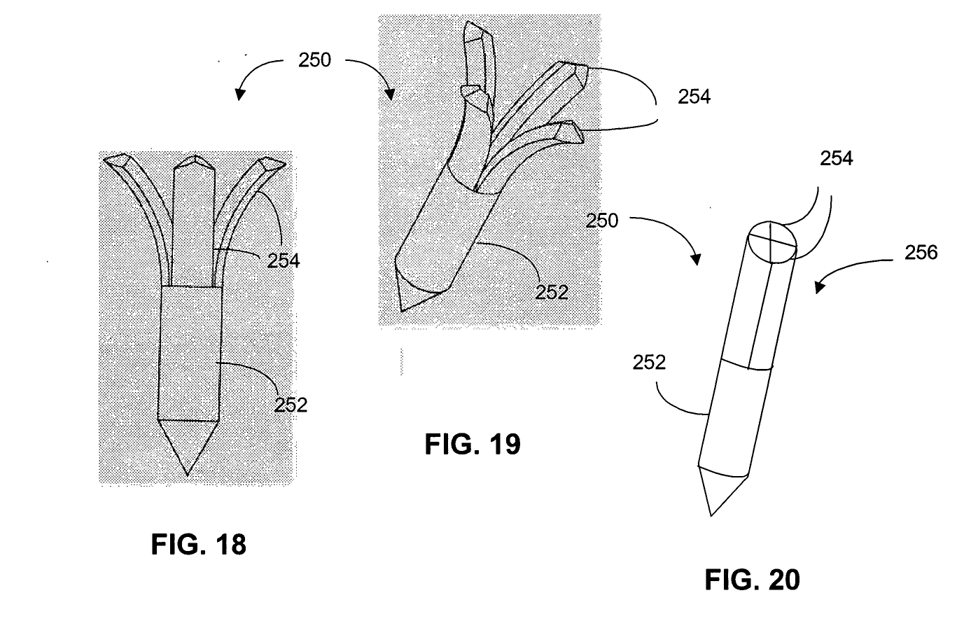

Referring now to FIGS. 18-20 there are seen, in accordance with an added embodiment of the present invention, schematic views of a expandable bone fastener generally referenced 250 having a generally cylindrical body referenced 252 and at least one pair of fastening projections referenced 254 formed from a shape memory alloy. In FIGS. 18-20, fastener 250 is shown as having two pairs of projections 254. While in an austenitic state, projections 254 remain in an open configuration as indicated in FIGS. 18 and 19.

The mechanism for utilizing a bone fastener 250 is substantially similar to that required for bone staples, as disclosed herein above in conjunction with FIGS. 14, 15 and 16. In order to locate bone fastener 250, a hole with a diameter to facilitate insertion, is drilled into the bone (not shown). Prior to insertion, fastener 250 is cooled so as to cause a transformation of the shape memory alloy to a fully martensitic state so that projections 254 are plastically deformable. As indicated in FIG. 20, projections 254 are drawn together so as to form a substantially cylindrical configuration generally referenced 256 when the alloy is in its martensitic state. Bone fastener 250 is then positioned in the drilled hole in the bone. Body heat warms fastener 250 to body temperature, causing projections 254 to deflect outwards against the inner surface of the hole, providing a resisting force thereto. The shape memory alloy of projections 254 is thereby transformed into an SRM state, as the stress-retained martensite attempts to transform into austenite. Projections 254 deflect outwards causing fastener 250 to be fastened into the bone.

Using SRM allows ease of deformation without the need for fastening projections 254 in a closed position and without the need for a special placement device. This contrasts with the use of an SIM alloy for a bone anchor, where the anchoring projections need to be forced into a closed elastic configuration prior to insertion and have to be inserted using a special placement device.

Stents

Carotid angioplasty and stenting are alternatives to surgery for the treatment of atherosclerotic carotid arteries, and randomized clinical trials. The biocompatibility and shape recoverability of shape memory alloys make them useful for this procedure.

Commonly, superelastic (pseudoelastic) behavior is used for self-expanding stents. The self-expanding stent (coil or mesh) diameter is preset to be somewhat larger than that of the target vessel. The opened stent is crimped or straightened, leading to a phase transformation to stress-induced martensite, restrained in a delivery system such as a catheter and then elastically released into the target vessel.

The main difficulties arising from using a SIM alloy stent are restraining the deformed stent in its metastable martensitic phase, and preventing it from regaining a preset shape prior to final insertion into a restraining means such as a catheter.

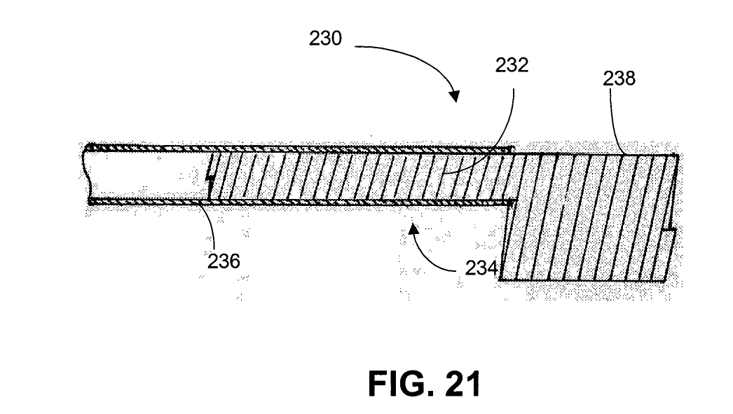

If an SRM element is used, the preparation prior to insertion is easily accomplished. Referring now to FIG. 21, in accordance with an embodiment of the present invention, there is seen a coil stent generally referenced 230 formed from a shape memory alloy wire referenced 232 in the shape of a helical coil. Coil stent 230 is cooled to a reduced temperature, below body temperature, when the shape memory alloy is transformed from an austenitic to a martensitic state. Stent 230 is easily deformed to an insertable size and shape generally referenced 234, so as to fit into a cooled delivery applicator or catheter referenced 236.

Coil 230 retains its insertable size and shape 234 without requiring any restraining instruments. It is easily inserted while cool into cooled catheter 236. This aspect is especially important when using long stents. The alloy transforms from its stable martensitic state to its metastable stress-retained martensitic state, when heated to an ambient temperature and in the presence of a restraining catheter. Subsequent insertion into a vessel is accomplished by pushing coil stent from catheter 236. Expansion occurs immediately to a preset size referenced 238 as stent 230 is released from catheter 236 and the alloy reverts to its austenitic state.

Vessel Filter

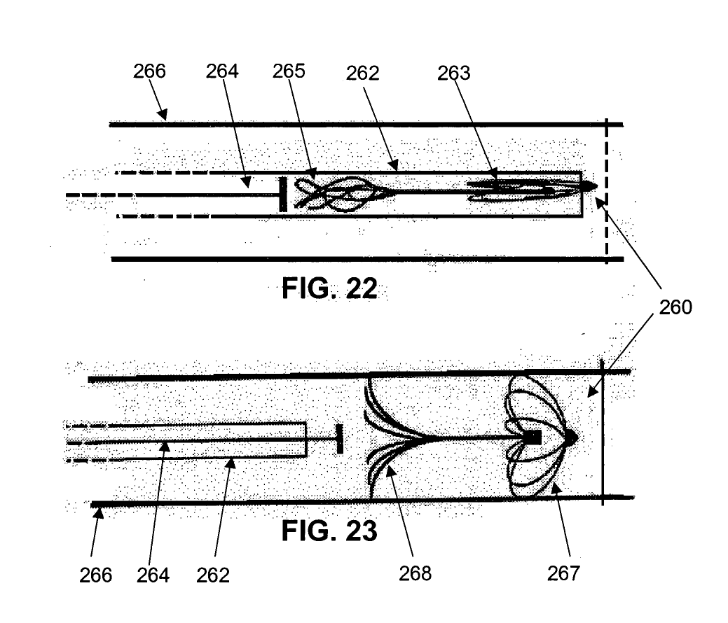

Referring now to FIGS. 22 and 23, there are seen, in accordance with an additional embodiment of the present invention, schematic views of a vessel filter generally referenced 260 prior to and after final installation respectively. After being cooled to an at least partially martensitic state, generally about 0° C., filter 260 is deformed so as to be insertable into a catheter referenced 262 equipped with a pusher device referenced 264. While in catheter 262, filter 260 is warmed. The restrictive force of catheter 262 prevents filter 260 from reverting to an austenitic state, and correspondingly to its original shape. The stable martensite of the alloy undergoes transformation to a stressed retained martensitic (SRM) state. Catheter 262 is introduced into a pre-selected blood-vessel referenced 266. By moving pusher 264 forward, filter 260 is ejected from catheter 262 into blood vessel 266. Upon_{[o]} unloading, the SRM state of the alloy of filter 260 transforms to its austenitic state. Primary 263 and secondary 265 elements expand to their original shape and lodge in blood vessel 266. Primary 263 and secondary 265 elements form primary and secondary supporting webs referenced 267 and 268 respectively. Any blood clots borne in the blood stream impinge against supporting webs 267 and 268 and are fragmented thereby.

Intrauterine Devices (IUD)

Application of SRM to IUDs is generally similar to that disclosed hereinabove in relation to vessel filters as shown in FIGS. 22 and 23.

Clamp

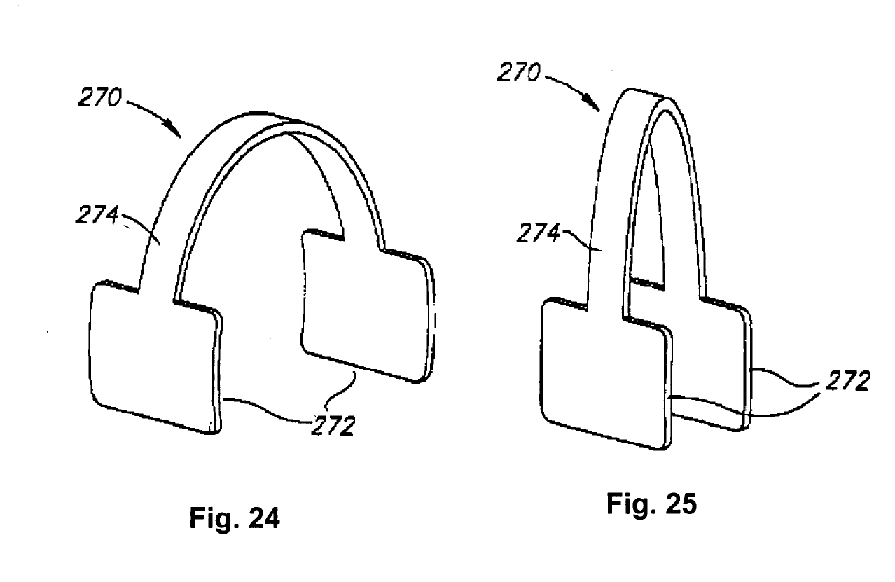

In accordance with a further embodiment of the present invention, reference is now made to FIGS. 24 and 25. FIGS. 24 and 25 show schematic perspective views of a clamp generally referenced 270 in an open and in a closed configuration, respectively. Prior to use, connecting portion referenced 274 is cooled so as to cause the shape memory alloy from which clamp 270 is constructed to transform to a plastic martensitic state. Clamp jaws referenced 272 are moved apart and retain this deformed shape as indicated in FIG. 24. After engaging jaws 272 over a tissue portion or portions (not shown), connecting portion 274 is warmed by body heat causing the alloy to begin to revert to an austenitic state. As the shape begins to revert to the closed configuration shown in FIG. 25, jaws 272 engage the selected tissue therebetween. The presence of the interposed tissue exerts a resisting force on clamp 270, specifically on connecting portion 274, preventing complete restoration of the original fully closed shape. This allows the martensite of the alloy to be transformed into stress retained martensitic (SRM), and, while the SRM transforms to an austenitic state, clamp 270 exerts a continuing clamping force on the engaged tissue. Alternatively, clamp 270 is restrained in a suitable applicator prior to use, and the resultant warming results in SRM formation.

Dental Implant

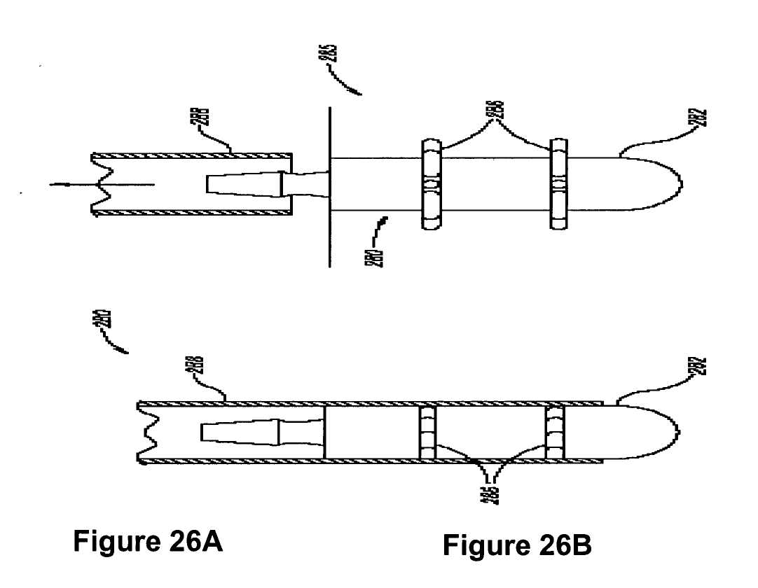

Referring now to FIGS. 26A and 26B there are seen, in accordance with another embodiment of the present invention, schematic views of a dental implant generally referenced 280. FIG. 26A shows the implant prior to implantation while FIG. 26B shows the implant after implantation into jawbone 285. Implant 280 includes a body portion referenced 282 and a plurality of projections referenced 286 formed of a shape memory alloy. When at body temperature, that is when dental implant 280 is implanted into jawbone 285, projections 286 are in an austenitic state and are configured to project radially outwards from body 282 as in FIG. 26. Prior to implantation (FIG. 26A), dental implant 280 is cooled so as to transform the alloy of projections 286 to a plastic martensitic state. As shown in FIG. 26A, projections 286 are folded circumferentially. Prior to implantation, projections 286 are inserted into a cooled holding tool referenced 288 so as to retain the projections in a martensitic state and in their folded configuration. Dental implant 280 is inserted into a selected jaw-bone 285 cavity in FIG. 26B and allowed to warm to body temperature. The alloy begins to revert to an austenitic state and folded projections 286 of FIG. 26A begin to revert to the extended projection 286 configuration of FIG. 26B. Projections 286 open outwards and come into engagement with the jawbone 285 cavity which applies a resisting force. The alloy transforms into stress retained martensitic (SRM) state and applies a continuing force to the bone 285 cavity so as to remain permanently engaged therein

Heart Valve Retaining Ring

Jervis, in U.S. Pat. No. 6,306,141, describes the use of a SIM ring to hold a sewing cuff to a body of an artificial heart valve. It is claimed that SIM alloys will provide the best alternative for this purpose. According to Jervis, the ring is expanded from its initial austenitic state with the transformation to SIM. As disclosed hereinabove in relation to FIG. 8, the stress required to strain an object in an austenitic state is several times higher than when the object is in a martensitic state. Alternatively, the ring is positioned about the valve body, heated above A_{f} and then cooled to its original temperature. This procedure causes the ring to engage the valve body to the heart.

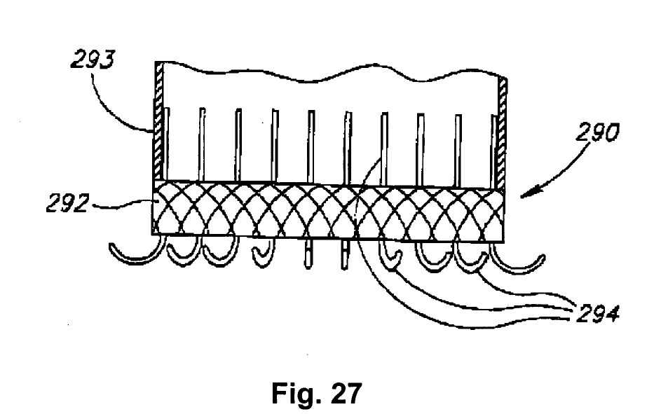

Using an SRM alloy does not require special heating of the ring. Body heat is sufficient to cause the requisite phase transformation. Referring now to FIG. 27, in accordance with another embodiment of the present invention, there is seen a schematic view of a shape memory alloy sewing ring 290 having spines (hooks) 294. Ring 290 is covered by a fabric seal (cuff) referenced 292, to prevent an infiltration between an artificial heart valve and a heart (not shown), utilizing a retaining ring (means) 293. Ring 290 is cooled to transform the alloy from which it is constructed from its austenitic to its malleable martensitic state so that hooks referenced 294 of sewing ring 290 are distortable to an open configuration. Thereupon retaining ring 293 is placed in position over sewing ring 290 and allowed or caused to warm to or above the original temperature. The heart valve provides a restraining means and exerts a resisting force against closure of ring 290, resulting in the formation of stress retained martensite (SRM) in the alloy of ring 290.

It will be appreciated by persons skilled in the art that the present invention is not limited by the drawings and description hereinabove presented. Rather, the invention is defined solely by the claims that follow.

1. A method for utilizing a deformable article of manufacture adapted to have selectable first and second predetermined configurations, the article being formed at least partly of a shape memory alloy, said method including the steps of
a) deforming the article under a deforming force from the first predetermined configuration to the second predetermined configuration while the shape memory alloy is, at least partially, in its stable martensitic state and at a first temperature;
b) applying a resisting force to the deformed article of manufacture using a restraining means,
c) heating the article from the first temperature to a second temperature in the presence of the resisting force, thereby transforming the alloy from its stable martensitic state to its metastable stress-retained martensitic state, while the article remains in its second configuration; and
d) removing the resisting force thereby to allow the alloy to transform to its austenitic state and the shape of the article to be restored substantially to the first configuration.

2. A method according to claim 1, wherein the article of manufacture is a medical device.

3. A method according to claim 1, further including the step of positioning the deformed article within the human body while the deformed article is restrained by the restraining means.

4. A method according to claim 3, wherein said step of heating is a step of automatically warming to body temperature when the article is positioned in or near the human body, body temperature being above the alloy's A_{f} temperature.

5. A method according to claim 1, further including the step of positioning the deformed article within the human body and wherein the restraining means is body tissue.

6. A method according to claim 5, wherein said step of heating is a step of automatically warming to body temperature when the article is positioned in or near the human body, body temperature being above the alloy's A_{f} temperature.

7. A method according to claim 1 further including the step of cooling prior to said step of deforming, said step of cooling including cooling the article to the first temperature such that the shape memory alloy, at least partially, transforms into its stable martensitic state.

8. A method according to claim 7, wherein said step of cooling includes cooling the article from the alloy's austenitic state to a state wherein the alloy is at least partially in its stable martensitic state.

9. A method according to claim 1 further including the step of heating the article of manufacture until A_{f} such that the shape memory alloy preserves its stable martensitic state.

10. A method according to claim 1 wherein said step of heating is a step of automatically warming to body temperature when the article is positioned in or near the human body, body temperature being above the alloy's A_{f} temperature.

11. A method according to claim 1 wherein said step of heating includes the step of heating to above the alloy's A_{f} temperature.

12. A method according to claim 1 wherein said step of removing is effected isothermally.

13. A method according to claim 1 wherein the first temperature is below Mₛ.

14. A method according to claim 1 wherein the first temperature is below Mₛ and the second temperature is above A_{f}.

15. A method according to claim 1 wherein the first temperature is below A_{f} and the second temperature is above A_{f}.

16. A method according to claim 1 wherein the restraining means in said step of applying is body tissue.

17. A method according to claim 1 wherein in said step of deforming a deformation is effected by a means for deforming which is the same means as the restraining means in said step of applying and the resisting force in said step of applying is substantially a continuation of the deforming force provided in said step of deforming employed to deform the article.

18. A method according to claim 1 wherein in said step of deforming a deformation is effected by a means for deforming which is a means different from the restraining means in said step of applying.

19. A method according to claim 18 wherein said restraining means is body tissue.

20. A selectably deformable article of manufacture adapted to have selectable first and second predetermined configurations, said article being formed at least partly of a shape memory alloy, wherein
a) said shape memory alloy is at least partially in a stable martensitic state and at a first temperature, thereby to facilitate deformation of said article from the first predetermined configuration to the second predetermined configuration; and
b) said shape memory alloy is further transformable from said stable martensitic state to a metastable stress-retained martensitic state, when heated to at least a second temperature in the presence of a predetermined resisting force, the resisting force impeding transformation of said shape memory alloy from said metastable stress-retained martensitic state to an austenitic state and thereby also impeding reversion of said article of manufacture from said second predetermined configuration to said first predetermined configuration.

21. A selectably deformable article of manufacture according to claim 20, wherein said first temperature is below Mₛ.

22. A selectably deformable article of manufacture according to claim 20, wherein said first temperature is below A_{f}

23. A selectably deformable article of manufacture according to claim 20, wherein said second temperature is above A_{f}.

24. A selectably deformable article of manufacture according to claim 20, wherein said stable martensitic state is attained by cooling the alloy to a first temperature below its Mₛ temperature from above its A_{f} temperature.

25. A selectably deformable article of manufacture according to claim 20, wherein said metastable stress-related martensite transforms to said austenitic state upon removal of the resisting force and the article reverts to its first configuration from its second configuration.

26. A selectably deformable article of manufacture according to claim 20, wherein said article of manufacture is a medical device.

27. A selectably deformed article according to claim 20, wherein said second temperature is substantially body temperature and A_{f} is below body temperature.

28. A selectably deformable article of manufacture according to claim 26, wherein said medical device is a surgical clip including a first and second length of a wire defining a pair of closed geometrical shapes, said shape substantially similar in configuration and size and having central openings, wherein said first and second lengths of wire fully overlap in a predetermined side-by-side registration, and at least an intermediate portion of said wire is formed of said shape memory alloy and is disposed between said first and second lengths of wire,
wherein said shape memory alloy is reversibly transformable from an austenitic state to a stable martensitic state, when cooled to a first temperature, thereby to facilitate deformation of said clip from said predetermined side-by-side registration to a predetermined open configuration; and
wherein said shape memory alloy is further transformable from said stable martensitic state to a metastable stress-retained martensitic state, when heated to at least a second temperature and in the presence of a predetermined resisting force, which impedes reversion of said shape memory alloy from said metastable stress-retained martensitic state to said austenitic state and which impedes reversion of said clip from said predetermined open configuration to said side-by-side registration.

29. A selectably deformable article of manufacture according to claim 26, wherein said medical device is an anastomosis ring for crimping adjacent intussuscepted organ wall portions against a generally tubular crimping support element having transversely formed end wall portions, so as to cause anastomosis between the organ wall portions, wherein said anastomosis ring includes a length of wire having a predetermined cross-sectional shape, formed of said shape memory alloy, said length of wire defining a closed substantially circular shape having a central opening and having overlapping end portions,
wherein said shape memory alloy is reversibly transformable from an austenitic state to a stable martensitic state, when cooled to a first temperature, thereby to facilitate deformation of said anastomosis ring to a predetermined open configuration; and
wherein said shape memory alloy is further transformable from said stable martensitic state to a metastable stress-retained martensitic state, when heated to at least a second temperature and in the presence of a predetermined resisting force, which impedes reversion of said shape memory alloy from said metastable stress-retained martensitic state to said austenitic state and which impedes reversion of said anastomosis ring from said predetermined open configuration to a predetermined crimping configuration.

30. A selectably deformable article of manufacture according to claim 26, wherein said medical device is a staple for bone fixation, formed of said shape-memory alloy, which includes:
a web having a first span length and a thickness;
two bending points, forming the end points of said web; and
two semicircular end sections, beginning from said bending points, having a preselected radius of curvature, a preselected angle of curvature, and a thickness which is substantially the same as said web thickness,
wherein said shape memory alloy is reversibly transformable from an austenitic state to a stable martensitic state, when cooled to a first temperature, to facilitate deformation of said staple to a predetermined open configuration, such that said angle of curvature is decreased to about 90° relative to said web so as to substantially straighten said semicircular end sections and to increase said span length to a preselected value, thereby to facilitate insertion of said staple into the bone; and
wherein said shape memory alloy is further transformable from said stable martensitic state to a metastable stress-retained martensitic state, when heated to at least a second temperature and in the presence of a predetermined resisting force, which impedes reversion of said shape memory alloy from said metastable stress-retained martensitic state to said austenitic state and which impedes reversion of said staple from said predetermined open configuration to a predetermined fastening configuration.

31. A selectably deformable article of manufacture according to claim 26, wherein said medical device is an expandable bone fastener for securing in a hole drilled in a bone, which includes:
a generally cylindrical elongate body having a substantially conical proximal end portion and a distal transverse end;
at least one deformable anchor arm formed of said shape-memory alloy fixably attached to said cylindrical body portion; and
fastening means disposed at apex of said conical proximal end portion;
wherein said shape memory alloy is reversibly transformable from an austenitic state to a stable martensitic state, when cooled to a first temperature, to facilitate deformation of said at least one anchor arm to a predetermined closed configuration; and
wherein said shape memory alloy is further transformable from said stable martensitic state to a metastable stress-retained martensitic state, when heated to at least a second temperature and in the presence of a predetermined resisting force, which impedes reversion of said shape memory alloy from said metastable stress-retained martensitic state to said austenitic state and which impedes reversion of said at least one anchor arm from said predetermined closed configuration to a predetermined expanded fastening configuration.

32. A selectably deformable article of manufacture according to claim 26, wherein said medical device is an expandable bone anchor for securing in a hole drilled in a bone, which includes:
a generally cylindrical elongate body having a substantially conical proximal end portion and a distal end;
at least one pair of deformable anchor projections formed of said shape-memory alloy fixably attached to said distal end of said cylindrical body portion; and
anchoring means disposed at apex of said conical proximal end portion;
wherein said shape memory alloy is reversibly transformable from an austenitic state to a stable martensitic state, when cooled to a first temperature, to facilitate deformation of said at least one pair of anchor projections to a predetermined closed configuration; and
wherein said shape memory alloy is further transformable from said stable martensitic state to a metastable stress-retained martensitic state, when heated to at least a second temperature and in the presence of a predetermined resisting force, which impedes reversion of said shape memory alloy from said metastable stress-retained martensitic state to said austenitic state and which impedes reversion of said at least one pair of anchor projections from said predetermined closed configuration to a predetermined expanded fastening configuration.

33. A selectably deformable article of manufacture according to claim 26, wherein said medical device is a stent for disposing in a human vessel so as to provide improved liquid circulation therethrough, said stent is formed of a shape memory alloy having a predetermined first configuration;
wherein said shape memory alloy is reversibly transformable from an austenitic state to a stable martensitic state, when cooled to a first temperature, to facilitate deformation of said stent to a predetermined second configuration which is smaller in size than said first configuration; and
wherein said shape memory alloy is further transformable from said stable martensitic state to a metastable stress-retained martensitic state, when heated to at least a second temperature and in the presence of a predetermined resisting force, which impedes reversion of said shape memory alloy from said metastable stress-retained martensitic state to said austenitic state and which impedes reversion of said stent from said predetermined second configuration to said predetermined first configuration.

34. A selectably deformable article of manufacture according to claim 33, wherein said predetermined resisting force is provided by a catheter.

35. A selectably deformable article of manufacture according to claim 33, wherein said stent is chosen from a group of stents consisting of a mesh stent and a coil stent.

36. A selectably deformable article of manufacture according to claim 26, wherein said medical device is an intrauterine device for disposing within a uterus, said device having a predetermined shape formed of said shape-memory alloy including anchoring means for attachment within the uterus,
wherein said shape memory alloy is reversibly transformable from an austenitic state to a stable martensitic state, when cooled to a first temperature, to facilitate deformation of said device and said anchoring means to a predetermined closed configuration; and
wherein said shape memory alloy is further transformable from said stable martensitic state to a metastable stress-retained martensitic state, when heated to at least a second temperature and in the presence of a predetermined resisting force, which impedes reversion of said shape memory alloy from said metastable stress-retained martensitic state to said austenitic state and which impedes reversion of said device and said anchoring means from said predetermined closed configuration to a predetermined expanded attaching configuration.

37. A selectably deformable article of manufacture according to claim 26, wherein said medical device is a heart valve retaining ring, wherein said heart valve retaining ring includes parts formed of said shape memory alloy, said parts defining a closed substantially circular shape having a central opening and having overlapping end portions,
wherein said shape memory alloy is reversibly transformable from an austenitic state to a stable martensitic state, when cooled to a first temperature, to facilitate deformation of said heart valve retaining ring to a predetermined opened configuration; and
wherein said shape memory alloy is further transformable from said stable martensitic state to a metastable stress-retained martensitic state, when heated to at least a second temperature and in the presence of a predetermined resisting force, which impedes reversion of said shape memory alloy from said metastable stress-retained martensitic state to said austenitic state and which impedes reversion of said heart valve retaining ring from said predetermined opened configuration to a predetermined contracted fastening configuration.

38. A selectably deformable article of manufacture according to claim 26, wherein said medical device is a clamp device for securing tissue, said clamp device includes:
a pair of clamping jaws fixably attached to a connecting portion formed of said shape-memory alloy;
wherein said shape memory alloy is reversibly transformable from an austenitic state to a stable martensitic state, when cooled to a first temperature, to facilitate deformation of said clamp device to a predetermined opened configuration; and
wherein said shape memory alloy is further transformable from said stable martensitic state to a metastable stress-retained martensitic state, when heated to at least a second temperature and in the presence of a predetermined resisting force, which impedes reversion of said shape memory alloy from said metastable stress-retained martensitic state to said austenitic state and which impedes reversion of said connecting portion from said predetermined opened configuration to a predetermined closed fastening configuration.

39. A selectably deformable article of manufacture according to claim 26, wherein said medical device is a blood vessel filter for fixably disposing within a major blood vessel thereby to fragment any blood clots flowing therethrough, said filter includes:
a) an elongate central axial support member having a first and second end;
b) a plurality of generally radial primary elements formed from a shape memory alloy wire exhibiting stress retained martensitic characteristics, said primary elements being fixably attached to said first end of said axial support member so as to form a primary supporting web; and
c) a plurality of generally radial secondary elements formed from a shape memory alloy wire exhibiting stress retained martensitic characteristics, said secondary elements being fixably attached to said second end of said axial support member so as to form a secondary supporting web;
wherein said shape memory alloy is reversibly transformable from an austenitic state to a stable martensitic state, when cooled to a first temperature, to facilitate deformation of said blood vessel filter to a predetermined closed configuration for insertion into a catheter; and
wherein said shape memory alloy is further transformable from said stable martensitic state to a metastable stress-retained martensitic state, when heated to at least a second temperature and in the presence of a predetermined resisting force, which impedes reversion of said shape memory alloy from said metastable stress-retained martensitic state to said austenitic state and which impedes reversion of said blood vessel filter from said predetermined closed configuration to a predetermined expanded fastening configuration.

40. A selectably deformable article of manufacture according to claim 26, wherein said second temperature is lower than body temperature and above the alloy's Af temperature.

41. A selectably deformable article of manufacture according to claim 26, wherein said second temperature is body temperature, body temperature being above the alloy's A_{f} temperature.
